(19) United States
(12) Patent Application Publication
(10) Pub. No.: US 2021/0290699 A1
(43) Pub. Date: Sep. 23, 2021

(54) GHRELIN SECRETION PROMOTOR

(71) Applicant: Meiji Co., Ltd., Chuco-ku (JP)

(72) Inventors: Akira Tamura, Odawara-shi, Kanagawa (JP); Satomi Koyama, Odawara-shi, Kanagawa (JP); Yukio Asami, Odawara-shi, Kanagawa (JP)

(73) Assignee: Meiji Co., Ltd., Tokyo (JP)

(21) Appl. No.: 16/316,078

(22) PCT Filed: Jul. 13, 2017
(86) PCT No.: PCT/JP2017/025506

§ 371 Date: Jan. 8, 2019

(30) Foreign Application Priority Data
Jul. 14, 2016 (JP) 2016-139372

(51) Int. Cl. A61K 35/747; A23C 9/123; A61K 9/00; A61P 1/14

(52) U.S. Cl. CPC A61K 35/747; A23C 9/1234; A61K 2035/115; A61P 1/14; A23Y 2220/37; A61K 9/0056

(57) ABSTRACT

Object of the invention is to provide a novel, safe, and side effect-free ghrelin secretion promoter which is capable of exhibit secreting effect of ghrelin present in human body in a form of food that is familiar and easy to be taken in. Hypophagia (loss of appetite) can be remarkably improved by a familiar, easy and safe mean by taking a given lactic acid bacteria per se or fermented milk prepared using the same as a ghrelin secretion promoter.

### TECHNICAL FIELD

The present invention relates to a ghrelin secretion promoter.

### BACKGROUND ART

Ghrelin is a peptide hormone that is predominantly secreted in stomach, and has been reported to have effects such as eating enhancement and enterokinesis-regulating effect, growth hormone-promoting effect, energy metabolism-regulating effect, cardiac function-enhancing effect, and anti-diabetic effect. Therefore, substances that promote the secretion of ghrelin have great utility since the promoted ghrelin secretion can improve loss of appetite (hypophagia). So far, milk constituents, plant extracts or Chinese herbal medicines have been reported to promote ghrelin secretion. On the other hand, there has never been reported about the promotion of ghrelin secretion by lactic acid bacteria. Accordingly, it was desired to achieve ghrelin secretion-promoting effect by more convenient method that is independent of medical products, etc., such as normal food intake prepared with lactic acid bacteria.

Previous examples include, for example, JP 2009-524640 A (Patent Literature 1) which describes that the secretion of ghrelin is regulated using Lactobacillus acidophilus, Lactobacillus salivarius, Lactobacillus curvatus etc. JP 2015-127338 A (Patent Literature 2) describes a composition comprising lactic acid bacteria of the genus Lactobacillus and ghrelin. JP 2015-205829 A (Patent Literature 3) and JP 2015-205830 A (Patent Literature 4) describe ghrelin secretion promoters which comprise skim milk or powdered skim milk as active ingredients. However, these literatures neither describe nor suggest that lactic acid bacteria, particularly Lactobacillus gasseri could promote the secretion of ghrelin and improve hypophagia.

### PRIOR ART REFERENCES

### Patent Literatures

Patent Literature 1: JP 2009-524640 A

Patent Literature 2: JP 2015-127338 A

Patent Literature 3: JP 2015-205829 A

Patent Literature 4: JP 2015-205830 A

### SUMMARY OF THE INVENTION

### Problems to be Solved by the Invention

So far, in order to improve loss of appetite (hypophagia) using ghrelin, it was necessary to practically force the subject to take in (or be administered) ghrelin or a known substance that induces ghrelin secretion. Thus, there has been a physical and/or psychological barrier to its intake.

In such background, the present invention is aimed to provide a novel, safe and side-effect-free ghrelin secretion promoter in a form that facilitates the intake that is capable of exhibiting an effect of inducing the secretion of ghrelin endogenously present in human body.

### Means to Solve the Problems

In order to solve the above-described problem, the inventors focused on fermented milk as a food that is familiar and can easily be taken, and carried out a detailed investigation for the functionality of the lactic acid bacteria to be used for preparing fermented milk. Consequently, we have found that certain lactic acid bacteria and fermented milk prepared using the same have a remarkable effect of inducing ghrelin secretion, and thereby completed the present invention.

Namely, the present invention is as follows.

[1] Ghrelin secretion promoter comprising lactic acid bacteria of the genus Lactobacillus as an active ingredient.
[2] The ghrelin secretion promoter according to [1], wherein the lactic acid bacteria of the genus Lactobacillus is Lactobacillus gasseri.
[3] The ghrelin secretion promoter according to [1] or [2], wherein the lactic acid bacteria of the genus Lactobacillus is Lactobacillus gasseri OLL2716 (FERM BP-6999).
[4] The ghrelin secretion promoter according to any one of [1] to [3], wherein the ghrelin secretion promoter is for a Helicobacter pylori-negative subject.
[5] Fermented milk comprising ghrelin secretion promoter according to any one of [1] to [4].

The present invention also encompasses the following inventions.

[6] Method for promoting ghrelin secretion comprising a step of making a subject to take fermented milk prepared using lactic acid bacteria of the genus Lactobacillus.
[7] The method for promoting ghrelin secretion according to [6], wherein the lactic acid bacteria of the genus Lactobacillus is Lactobacillus gasseri.
[8] The method for promoting ghrelin secretion according to [6] or [7], wherein Lactobacillus gasseri is Lactobacillus gasseri OLL2716 (FERM BP-6999).
[9] Use of lactic acid bacteria of the genus Lactobacillus in the production of a ghrelin secretion promoter.
[10] The use according to [9], wherein the lactic acid bacteria of the genus Lactobacillus is Lactobacillus gasseri.
[11] The use according to [9] or [10], wherein Lactobacillus gasseri is Lactobacillus gasseri OLL2716 (FERM BP-6999).
[12] Lactic acid bacteria of the genus Lactobacillus for use in the production of a ghrelin secretion promoter.
[13] Lactobacillus gasseri OLL2716 (FERM BP-6999) which is a lactic acid bacteria of the genus Lactobacillus for use in the production of a ghrelin secretion promoter.

### Effects of the Invention

According to the present invention, an effect of promoting ghrelin secretion can be achieved and loss of appetite (hypophagia) can significantly be improved by taking in certain lactic acid bacteria or fermented milk prepared by the same. In particular, fermented milk is a familiar and well-experienced food which is easy and safe to be taken and thus capable of contributing to the improvement in hypophagia.

### BRIEF DESCRIPTION OF DRAWINGS

FIG. 1 A graph showing the result of the comparison of the concentration of the active form of ghrelin in blood between the control group and LG21 yogurt group in Example 1. Vertical bars indicate standard deviations within each group. There was a statistically significant difference between the control group and LG21 yogurt group at <5% significant level.

FIG. 2 A graph showing the result of the comparison of ghrelin gene expression level in stomach tissue between the control group and LG21 yogurt group in Example 1. Vertical bars indicate standard deviations within each group. There was a statistically significant difference between the control group and LG21 yogurt group at <5% significant level.

FIG. 3 A graph showing the result of the comparison of ghrelin gene expression level in stomach tissue in the control group, 1 hour after the application of LG21 viable cells, and 2 hours after the application of LG21 viable cells in Example 2. Vertical bars indicate standard deviations within each group. There were statistically significant differences between the control group and 2 hours after the application of LG21 viable cells, and between 1 hour after the application of LG21 viable cell and 2 hours after the application of LG21 viable cells at <5% significant level.

### DESCRIPTION OF EMBODIMENTS

In the present invention, lactic acid bacteria may be used as they are. In the context of the present invention, “lactic acid bacteria” collectively refers to all those taxonomically identified as lactic acid bacteria, which are not limited by bacterial species or strains. Lactic acid bacteria may sometimes be classified as plant or animal origin. Nevertheless, the lactic acid bacteria that can be used in the present invention may be either plant or animal origin. In the present invention, one or more bacterial strains selected from lactic acid bacteria of the genus Lactobacillus are favorable because they have a sufficient experience as food including fermented milk such as yogurt. Furthermore, among the lactic acid bacteria of the genus Lactobacillus, it is preferably Lactobacillus gasseri, and further preferably, Lactobacillus gasseri OLL2716 (FERM BP-6999).

Here, “Lactobacillus gasseri OLL2716” has been deposited to an international depositary authority based on the Budapest Treaty, to the International Patent Organism Depository of the National Institute of Advanced Industrial Science and Technology (central 6th, 1-1 Higashi 1-chome Tsukuba city, Ibaraki, Post Code 305-8566, Japan) under the deposit number FERM BP-6999 dated May 24, 1999. The present deposited strain was transferred from national deposition (original deposition) of FERM P-17399 dated May 24, 1999 to the international depositary authority on Jan. 14, 2000 under the Budapest Treaty.

Note that, as described in Budapest Notification No. 282 (http://www.wipo.int/treaties/en/notifications/budapest/treaty_budapest_282.html), NITE Patent Microorganisms Depositary, National Institute of Technology and Evaluation (IPOD, NITE) has succeeded patent microorganism depositary services from said the International Patent Organism Depository of the National Institute of Advanced Industrial Science and Technology (IPOD, AIST). Accordingly, the present deposited strain has been deposited to National Institute of Technology and Evaluation (IPOD, NITE) (Room 120, 2-5-8, Kazusakamatari, Kisarazu-shi, Chiba, Post Code 292-0818, Japan) (FERM BP-6999).

In another embodiment of the present invention, fermented milk may be prepared using lactic acid bacteria. According to the present invention, the lactic acid bacteria of the genus Lactobacillus of the present invention can be taken in without stress in a form of fermented milk as compared to in a case of taking in lactic acid bacteria per se thanks to the moderate and fresh sourness of the fermented milk. Methods of preparing fermented milk include, for example, a method comprising sterilizing raw milk, cooling it down, adding lactic acid bacteria as a starter, and fermenting the mixture at a fermentation temperature and time to obtain a predetermined lactic acid acidity. An exemplary lactic acid acidity is, for example, between 0.6 and 1.2 wt %. An exemplary fermentation temperature is, for example, between 40 and 45° C. An exemplary fermentation time is, for example, between 2 and 12 hours.

“Fermented milk” in the present invention means material obtained by fermenting milk. “Fermented milk” includes, for example, “fermented milk”, “lactic acid bacteria beverage”, “milk beverage” and “natural cheese” or the like, which are defined by Ministerial Ordinance on Milk and Milk Products Concerning Compositional Standards, etc., (Japanese Ministry of Health, Labour and Welfare Ordinance) but not limited thereto. For instance, fermented milk refers to “fermented milk” defined by Ministerial Ordinance on Milk and Milk Products Concerning Compositional Standards, etc., namely, milk such as raw milk, cow's milk, special milk, raw goat's milk, sterilized goat's milk, raw sheep's milk, composition modified milk, low-fat milk, fat-free milk and processed milk, or milk and the like containing milk solids-not-fat content which is equivalent to the above, which are fermented with lactic acid bacteria or yeast and made either solid (hard-type), paste (soft-type) or liquid (drink-type), optionally frozen, but not limited thereto. In the fermented milk of the present invention, the concentration of milk solids-not-fat content ranges, for example, preferably between 4.0% and 12.0%, more preferably between 6.0% and 10.0%, and further preferably between 7.0% and 9.0%. The concentration of milk fat is, for example, preferably between 0.2% and 4.0%, more preferably between 0.3% and 3.0%, and further preferably between 0.4% and 2.0%.

Typical examples of fermented milk include yogurt. In the present invention, “yogurt” includes such as, for example, plain yogurt, hard-type yogurt (set-type yogurt), soft-type yogurt, drink-type yogurt. In the present invention, drink-type yogurt is particularly preferred in view of the ease of drinking in the case of loss of appetite (hypophagia).

From the aspect of usability, the fermented milk of the present invention is preferably in a form of individual packages each containing an amount appropriate for a single intake so that it can appropriately and easily be taken. Although there are individual differences and variations depending on the level of hypophagia to be improved, the amount appropriate for a single intake in fermented milk containing 8.0% of milk solids-not-fat content is, for example, preferably between 50 mL and 200 mL, more preferably between 80 mL and 150 mL, and further preferably between 100 mL and 120 mL per one intake. Alternatively, it is preferably between 50 g and 200 g, more preferably between 80 g and 150 g, further preferably between 100 g and 120 g per one intake.

In the present invention, the “form of an individual package” encompass all forms including general packaging forms such as, for example, a container with a lid, a bottle with a cap, an individual packing bag, a pouch and a tube and the like. In the present invention, the use can be made clear by, for example, describing the uses, efficacy and method for intake of the product on the package including each or multiple of individual packages, and/or by adding a material of the description within the package, and/or by notifying a separate material of the description such as a pamphlet.

In the present invention, an effective amount (intake) of the active ingredient, i.e., the number of lactic acid bacteria for human per one day is preferably between 2×10⁷ and 5×10¹⁰, more preferably between 5×10⁷ and 5×10¹⁰, further preferably between 1×10⁸ and 5×10¹⁰, yet more preferably between 5×10⁸ and 5×10¹⁰, furthermore preferably between 5×10⁸ and 2×10¹⁰.

The fermented milk of the present invention provides a sufficient effect with single intake. Nevertheless, in view of increasing the effect of promoting ghrelin secretion, it is preferably continuously taken for 4 weeks or more, 6 weeks or more, 8 weeks or more, 10 weeks or more, or 12 weeks or more, and more preferably continuously taken for 24 weeks or more, and further preferably continuously taken for 36 weeks or more. Besides, the fermented milk of the present invention has a sufficient experience as food and can safely be taken, and therefore the upper limit of the duration for intake is not particularly limited and can be continued permanently. Nevertheless, if the upper limit is set by constraint, it is, for example, up to 120 weeks, up to 100 weeks, up to 80 weeks, or up to 60 weeks.

Moreover, according to the present invention, a commercially available product containing the lactic acid bacteria in an embodiment of the present invention may conveniently be employed. For instance, in the case of Lactobacillus gasseri OLL2716 (FERM BP-6999), “Meiji Probio yogurt LG21” manufactured and sold by Meiji Co., Ltd. may conveniently be employed. This commercially available product may be taken in as it is or may further be processed. The lactic acid bacteria in an embodiment of the present invention can be taken together with other ingredients that can be taken, which are not limited, and, for example, ingredients related to milk can suitably be used. The ingredient related to milk means a composition comprising milk or a milk constituent prepared by processing milk, including all ingredients containing milk constituents such as, for example, raw milk (e.g., cow's milk), recombined milk (e.g., powdered milk, cream and butter), fermented milk (e.g., yogurt, cheese), products prepared from milk (e.g., whey, casein, lactose, whey minerals, permeates), and their origins or forms are not particularly limited.

Besides, Lactobacillus gasseri OLL2716 has been known as a lactic acid bacteria having a high eradication ability against Helicobacter pylori in human stomach (e.g., Japanese Patent Publication No. JP 4509250 B). Nevertheless, the effect of the present invention to promote ghrelin secretion can be obtained irrespective of the eradication of Helicobacter pylori. Accordingly, the present invention may be used for a Helicobacter pylori-positive subject, or may also be used for a Helicobacter pylori-negative subject.

### EXAMPLES

Hereinbelow, the present invention is more specifically illustrated based on examples. Note that these examples are not to limit the present invention.

### Example 1

Twelve day mice (Japan SLC, Inc., 4 weeks old, male) were acclimatized for a period of 1 week and then divided into two groups: Control group and LG21 yogurt group. In the Control group, AIN-93M feed (CLEA Japan, Inc.; composition: corn starch 46.5692%, milk casein 14.0%, pregelatinized corn starch 15.5%, granulated sugar 10.0%, refined soybean oil 4.0%, cellulose powder 5.0%, minerals-mix (AIN-93M-MX) 3.5%, vitamins-mix (AIN-93VX) 1.0%, L-cystine 0.18%, choline bitartrate 0.25%, tert-butylhydroquinone 0.0008%) was given for 4 weeks. In the LG21 yogurt group, AIN-93M feed mixed with LG21 yogurt (“Meiji Probio yogurt LG21” being manufactured and sold by Meiji Co., Ltd. which comprises Lactobacillus gasseri OLL2716 (FERM BP-6999)) for 4 weeks such that the dosage would be 1.11 g/day. After 4 weeks, the mice were dissected to collect blood and stomach tissue.

The blood was treated with EDTA, then one tenth volume of hydrochloric acid (1 mol/L) was added and the concentration of the active form ghrelin in blood was measured using Active Ghrelin ELISA kit (LSI Medience Corporation). Furthermore, cDNA was prepared from collected stomach tissue using PrimeScript RT regent kit (Takara Bio Inc.), and ghrelin gene expression level in stomach tissue was measured using SYBER Premix Ex Tagil (Takara Bio Inc.) and Thermal cycler Dice (Takara Bio Inc.).

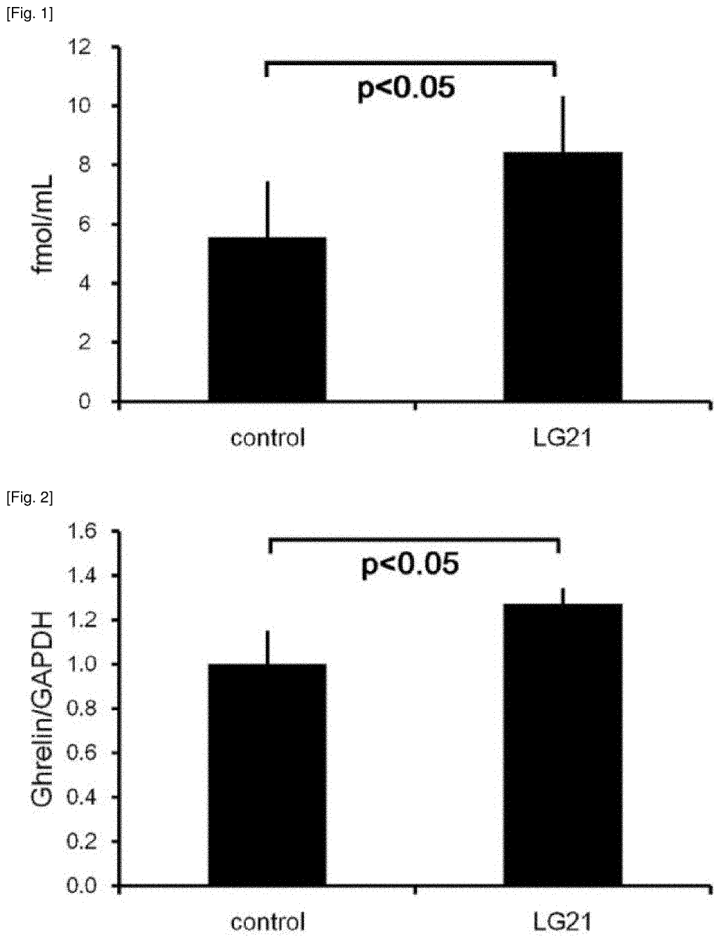

The result of the measurement of the concentration of the active form ghrelin in blood is shown FIG. 1, ghrelin gene expression level in stomach tissue in FIG. 2. In FIG. 1, the concentration of the active form ghrelin in blood was significantly higher in LG21 yogurt group as compared to the control group. Moreover, in FIG. 2, ghrelin gene expression level in stomach tissue was significantly higher as compared to the control group. Thus, it was suggested that LG21 yogurt comprising Lactobacillus gasseri OLL2716 (FERM BP-6999) has an effect of promoting ghrelin secretion.

### Example 2

Fifteen ICR mice (Japan SLC, Inc., 10 weeks old, male) were acclimated for a period of 1 week and then divided into 3 groups. After 5 hours of fasting, one group was designated as control group, 500 μL of physiological saline was administrated, dissected to collect stomach tissue. To the other two groups, 500 μL each of physiological saline containing 2×10¹⁰ cfu/mL of viable Lactobacillus gasseri OLL2716 (FERM BP-6999) cells was administered, dissected 1 to 2 hours after administration to collect stomach tissue. Ghrelin gene expression level in stomach tissue was measured by a method similar to Example 1.

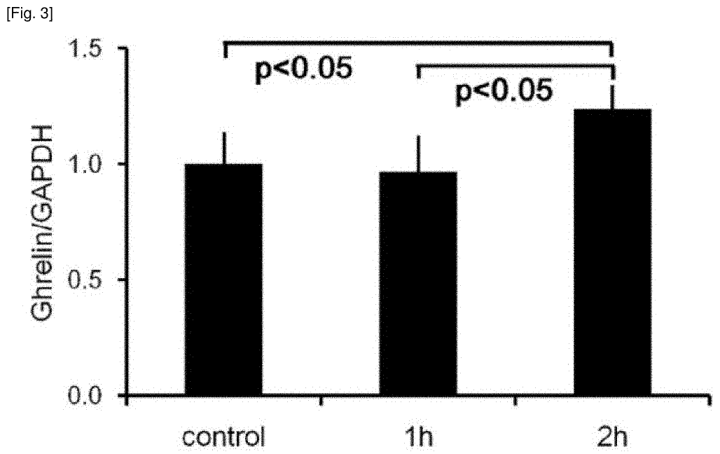

The results are shown in FIG. 3. At 2 hours after administrating viable Lactobacillus gasseri OLL2716 (FERM BP-6999), ghrelin gene expression level in stomach tissue was significantly higher as compared to the control group. Thus, it was suggested that the viable Lactobacillus gasseri OLL2716 (FERM BP-6999) has an effect of promoting ghrelin secretion.

### INDUSTRIAL APPLICABILITY

According to the present invention, an effect of promoting ghrelin secretion can be obtained and hypophagia (loss of appetite) can significantly be improved by taking in the lactic acid bacteria per se or fermented milk prepared using the same. Particularly, fermented milk is a familiar and well-experienced food which is easy and safe to be taken and thus capable of greatly contributing to the improvement in hypophagia.

1. A method for promoting ghrelin secretion comprising, the method comprising a subject taking or being made to take lactic acid bacteria of the genus Lactobacillus as an active ingredient.

2. The method according to claim 1, wherein the lactic acid bacteria of the genus Lactobacillus is Lactobacillus gasseri.

3. The method according to claim 1, wherein the lactic acid bacteria of the genus Lactobacillus is Lactobacillus gasseri OLL2716 (Deposit Accession number FERM BP-6999).

4. The method according to claim 1, wherein the subject is Helicobacter pylori-negative subject.

5. (canceled)

6. The method according to claim 1, wherein the lactic acid bacteria is comprised in yogurt.

7. The method according to claim 1, wherein the subject has hypophagia.
